(19) United States
(12) Patent Application Publication
(10) Pub. No.: US 2019/0351202 A1
(43) Pub. Date: Nov. 21, 2019

(54) A CAPSULE AND A SYSTEM THEREOF

(71) Applicant: MELCAP SYSTEMS LTD., HAIFA (IL)

(72) Inventors: Alexander Melamud, Haifa (IL); Boris Voznesensky, Raanana (IL); Rita Zucker, Haifa (IL); Igor Koman, Tel Aviv (IL)

(21) Appl. No.: 16/472,897

(22) PCT Filed: Jan. 11, 2018
(86) PCT No.: PCT/IL2018/050043

§ 371 Date: Jun. 24, 2019

(30) Foreign Application Priority Data
Jan. 12, 2017 (IL) 250090

(51) Int. Cl. A61M 31/00; A61B 1/04; A61F 5/00

(52) U.S. Cl. CPC A61M 31/002; A61B 1/041; A61F 5/0026; A61M 2205/6054; A61M 2230/65; A61M 2230/208; A61M 2205/3523; A61M 2205/0233; A61M 2205/0288; A61M 2210/106; A61M 2210/1064; A61M 2210/1053; A61M 2205/8206

(57) ABSTRACT

The present invention discloses a capsule configured for controllably remaining within a patient's body for a certain period of time as well as a system comprising the capsule thereof. The invention also discloses a capsule being configured and operable for electrical stimulation, as well as a system comprising the capsule thereof.

### TECHNOLOGICAL FIELD

The present invention relates to a capsule and system for treatment of obesity, overweight problems, gastroparesis, gastroesophageal reflux disease (GERD) and other diseases.

### BACKGROUND ART

References considered to be relevant as background to the presently disclosed subject matter are listed below:
US 2002/198470
US 2005/183733
US 2006/142794
US 2007/156248
US 2008/107732
US 2008/281374
US 2008/281375
US2011/208270
US 2014/107726

Acknowledgement of the above references herein is not to be inferred as meaning that these are in any way relevant to the patentability of the presently disclosed subject matter.

### BACKGROUND

Obesity is a major health problem in developed countries. In the United States, the complications of obesity affect nearly one in five individuals at an annual cost, in the US alone, of approximately $147 billion. Except for rare pathological conditions, weight gain is often directly correlated to overeating.

Presently there are different techniques and treatments for obesity, which include Endoscopic Bariatric Metabolic Therapies (EBMT). This consists of Intragastric Balloons (IGB), Transpyloric Shuttle, Aspiration Therapy, Plication and Suturing, Metabolic and Bariatric Surgery (MBS), Duodenal Mucosal Resurfacing, and others.

For example IGB, properly placed and sized, provide the patient with a feeling of satiety after ingesting only a small amount of food. Typically, the individual's caloric intake is thus diminished due to the subjective feeling of fullness. There are a number of available volume-occupying devices, many of such which must be introduced using surgical or other complex gastric procedures.

IGBs have been in clinical use for several years. Their success in the treatment of certain individuals with morbid obesity is well accepted.

Although diets, diet pills, and other weight-reducing plans may be effective for a short period of time, studies have shown that over 96 percent of those who lose as much as 100 pounds, tend to regain that lost weight, plus additional pounds, within 3 years. Known methods to successfully treat the morbidly obese are invasive and permanent. For example, surgical procedures such as gastric bypass or vertical banded gastroplasty have been used to reduce the size of the stomach or decrease absorption in the intestines of severely obese patients. However, these surgeries carry significant risks inherent in all invasive surgeries, and they cannot be modified or reversed.

More recently, systems and methods have been developed for reversibly treating obesity by stimulating the vagus nerve to produce a feeling of satiety, which leads to weight reduction. Side effects associated with implantable systems include risks associated with surgery, including bleeding, infection, and pain. Other problems associated with the implantation of an implanted nerve stimulation device is Neurostimulator migration leading to secondary surgery, pain or seroma at site of the Neurostimulator, perforation of the stomach wall, migration of leads, and an allergenic or immune system response to implanted materials. Implantable vagus nerve stimulation systems have also been used to control seizures in patients.

Electronically controlled ingestible capsules can be used to provide therapeutic treatment during traversal of the gastrointestinal alimentary tract. The capsule can be provided with an electronically controlled medicament delivery system. The capsule is moved by the peristaltic movement of the muscles along the gastrointestinal tract. An electronically controlled capsule can be programmed or controlled to deliver or dispense a medicament according to a dispensing timing pattern while traversing through the gastrointestinal tract. The electronically controlled capsule can include control and timing circuitry for controlling the opening and closing of a valve or hatch according to the desired dispensing timing pattern for dispensing a medicament stored within a medicament reservoir of the capsule.

Gastric retention systems are prepared in different ways, such as floating, bioadhesive, swelling and expanding, or high density types. Systems that are capable of swelling and being retained in the gastric environment give a feeling of satiety and can be used in controlling appetite, and therefore obesity.

Another subject which has undergone much study is the control of drug delivery. Such control may be spatial, temporal, or both. Controlled drug delivery systems deliver drugs to the body so as to establish therapeutically effective drug blood levels of the active ingredient, and, once these blood levels are achieved, they continue to maintain constant blood levels for long durations by delivering the drug to the body at a slow rate. By avoiding peaks and troughs in blood levels associated with conventional dosage forms, controlled drug delivery systems lower the incidence of adverse effects or side effects. Very importantly, controlled drug delivery systems reduce the frequency of dosing leading to convenience to the patient in terms of dosing and compliance to the specified dosage regimens.

It is generally known that the rate at which an oral controlled drug delivery system delivers the drug into the gastrointestinal fluids may not the same as the rate at which it releases the drug into a test aqueous fluid, because the gastrointestinal fluid's pH and composition change with the specific location of the drug delivery system in the gastrointestinal tract i.e. from the stomach to the colon, fasted versus fed state, type and amount of food ingested, and also due to variations in these factors from individual to individual. In addition, the drug may not be absorbed in the same manner and propensity as it moves from the stomach to the colon. Some drugs have an “absorption window” i.e. they are absorbed only from the upper parts of the gastrointestinal tract, whereas there are others whose absorption from the colon is not uniform or complete. Thus, the location of the controlled drug delivery system in the gastrointestinal tract, as well as the rate at which the controlled drug delivery system moves from the stomach to the colon, represent important factors that need to be considered in the design of an oral controlled drug delivery system. It is thus known to those skilled in the art that an oral controlled delivery system should be designed not only with control over the rate at which it releases the drug over the drug delivery time period (temporal control), but also control over the location from which it is delivered (spatial control). Such spatial control can be achieved by prolonging the period of retention of the system in the stomach. Gastric retention systems are also beneficial when the drug is effective locally in the stomach. Drugs absorbed in the upper part of the gastrointestinal tract may exhibit variability in absorption due to inter and intra-individual variability in gastric emptying and gastrointestinal motility. This variation in absorption is addressed partly by a gastric retention drug delivery system and may be further addressed by administering a dosage form comprising the drug, such that a part of the drug is available as immediate release, and a part is available as sustained or controlled release.

### GENERAL DESCRIPTION

The present invention relates inter alia to a novel capsule to be ingested or inserted in the human body via the rectum by a subject for obesity treatment and that controllably remains within a patient's body for a certain period of time, and to a system comprising inter alia the capsule thereof. According to a broad aspect of the present invention there is provided a capsule to be ingested by a subject or inserted in the human body via the rectum comprising: a cavity having an outer surface; wherein at least a portion of the outer surface is at least partially made with at least one dissolving material being configured to disintegrate at a certain region of the gastrointestinal tract, thereby disintegrating at least a portion of the outer surface at least one expandable element located within the cavity in a non-expanded state and configured to expand to a desired three-dimensional dimension; wherein the at least one expandable element is attached to a portion of the cavity to thereby enable retention of the capsule at the certain region, wherein the at least one expandable element is made of at least one material being configured for being evacuated after a certain period of time to thereby enable evacuation of the capsule outside the gastrointestinal tract; and a folded network structure having openings, the network structure being configured for holding a plurality of the at least one expandable element placed therein, a dimension of the elements being higher than a dimension of the openings, such that in a folded state the elements are not able to escape from the network structure, wherein upon disintegration of at least a portion of the outer surface, the network structure unfolds and the expandable elements expand to a desired three-dimensional dimension, wherein at least one of the folded network structure and the expandable elements is at least partially made of polymer-based material. The capsule of the present invention provides a long gastric retention of about several hours or even several days. The term “gastric retention” may refer to the maintenance or holding of a pharmaceutical in the stomach, for a time longer than the time it would have been retained in the stomach when delivered in a free form or within a gastro-intestinal delivery vehicle which is not considered gastroretentive. Gastro-retentivity may be characterized by retention in the stomach for a period that is longer than the normal emptying time from the stomach, such as longer than about 2 hours, in some cases longer than about 3 hours, and in many cases more than about 4, 6, 8 or 10 hours. Gastro-retentivity typically means retention in the stomach for a period of time of about 3, 4, 6, 8, 10, or at times 18 hours, even up to about 21 hours or longer. Gastro-retentivity may also mean retention in the stomach for a predetermined time period of at least 4, 6, 8, 10, 12, and 18 hours.

It should be noted that swelling to a large dimension is a significant factor in gastric retention of the capsule. Solids having a size less than 5 to 7 mm show delayed gastric emptying in fed conditions but they can still be emptied from the stomach because their size is smaller than the pyloric sphincter. Even floating systems of a size less than 5 to 7 mm can be emptied if the patient is in a supine position. The mean resting pyloric diameter is approx. 13+7 mm and it has been reported that dosage forms with a size of approx. 12-18 mm diameter in their expanded state would generally be excluded from the passage of the pyloric sphincter. The capsule of the present invention is capable of retaining this size in the gastric tract for long periods under agitational conditions created by gastric motility. It should be noted that, generally, in order to “hold” subjects in a stomach and not to be close to the pyloric area to prevent its blockage, these subjects (the capsule in the present invention) need to have spatial dimensions of 20-30 mm at least in two axes. The combination of increase in size and floatation results in increased gastric retention of the capsule. As used herein, the dimension of the capsule is suitable “to be ingested by a subject or inserted in the human body via the rectum”. The dimension of the capsule may be any size and/or shape of an encapsulated dosage form that is capable of being swallowed by either a human or an animal.

The expandable element is an element which can enlarge its volume while in contact with gastric fluid. The expandable element may be a self-expandable element such as a stent-like element or beads having shape memory property. The element may be attached in between the two opposite ends of the capsule, on the top or down side of the capsule or at any other possible empty spaces not occupied by electronics or power sources, or by flexible connection such as biodegradable or synthetic wires/sutures.

In a non-limiting example, the network structure may have a tubular shaped, disc shape or any other shaped element having a selected thickness and openings. The network structure may be weaved, braided or made by any other method applied for medical grade sutures. It may also be made by using any fiber production method such as electrospinning. The network structure may be made from biodegradable or non-degradable material organic or biological materials or a medical textile made from porous material.

In a specific and non-limiting example, the network structure is made of two types of textile/suture: one non-dissolvable, and the second dissolvable. Non-dissolvable material is closed by dissolvable sutures. The polymer-based material may be capable of being transformed to at least one of gel, hydrogel and suspension upon exposition with gastric juice. Swelling and transformation of the polymer into gel enables the retention of the expandable elements within the network structure. In a case of non-dissolvable material, after a specific period of time, the sutures are dissolved, the network itself unfolds, and the gel is freed and leaves the stomach naturally. The rest of the network leaves the stomach as well. In a case of dissolvable material, all or part of the network is dissolved, and the gel is freed and leaves the stomach naturally. The polymer-based material and network of both types may have at least one of the following properties: conductive and magnetic.

In some embodiments, the network structure is at least partially made of at least one of stretchable material, conductive material and biological sutures. The network may then be configured as an electrode used for transfer of at least one electrical signal at at least one region of the gastrointestinal (GI) tract. The network structure may comprise at least two different materials configured to evacuate the gastrointestinal tract after a certain period of time. At least one of the materials may be configured to dissolve at a certain region of the gastrointestinal tract to thereby enable escape of the expandable elements. The openings of the network structure may have dimensions of different sizes.

In some embodiments, the capsule may comprise at least two electrodes for applying at least one electrical signal at at least one region of the gastrointestinal (GI) tract in which the capsule passes through, the electrodes being located at the outer surface.

In some embodiments, the expandable element may be coated by an electrically conductive material (such as copper, Titanium, silver, gold and other metals with good conductive properties and certificated for use in medical implantable devices) being configured and operable as electrodes for applying at least one electrical signal in at least one region of the gastrointestinal (GI) tract.

In some embodiments, the capsule comprises a power supply, which may be a direct current power source, an alternating current power source, a battery, a primary electrochemical cell, a rechargeable electrochemical cell, a mechanical power source, alternative power sources or any combination thereof. The alternative power sources may include a voltaic cell that is sustained by the acidic fluids in the stomach, e.g. to use zinc and copper electrodes, located on the cavity of the capsule and/or energy harvesting module, using human body heat or biomechanical energy harvesting circuits.

In some embodiments the power supply may be actively activated. To this end, the power supply may be normally closed. The initialization stage in which the power supply is activated may be performed when the capsule is in the GI tract, for example by using the electrodes or by using a magnetic sensor activating a switch.

In some embodiments, the cavity comprises a compartment storing the expandable element(s).

In some embodiments, the at least one electrical signal applied by the at least two electrodes is configured for at least one of (1) measuring properties of the at least one region, (2) stimulating the at least one region and (3) delivering a drug substance to the at least one region.

In some embodiments the electrodes are configured and operable to stimulate a region in the gastrointestinal tract, block muscle contraction or neurostimulation for vagal block therapy, which may delay or stimulate activity in the intestine, or hold the capsule for a longer duration in the stomach and/or intestine.

In some embodiments, the at least one material of the at least one expandable element is configured for disintegration after a certain period of time.

In some embodiments, at least a portion of the outer surface is at least partially made with at least one dissolving material being configured to disintegrate at a certain region of the gastrointestinal tract upon exposition with gastric juice, thereby disintegrating at least a portion of the outer surface, such that after a certain period of time in the stomach the expandable element is able to expand itself in the stomach at a desired three-dimensional dimension, such that the capsule is controllably maintained at a certain (e.g. fixed) location for a certain period of time. In this way, the capsule can controllably be retained in the stomach.

In some embodiments, the attachment between the expandable element and the capsule is configured to disintegrate after a certain period of time.

In some embodiments, the capsule comprises a camera configured for controllably imaging any desired region of the gastrointestinal tract, such that the location of the capsule can be controlled.

In some embodiments, the at least one expandable element is at least partially coated with a protective layer to thereby prevent injury to the gastrointestinal tract.

In some embodiments, the capsule further comprises one or more gas generating elements (e.g. effervescent elements) placed within the network structure configured and operable to produce gas when placed in contact with fluid and to thereby increase inflation of the expandable elements.

In some embodiments, the capsule further comprises a filler placed within the network structure configured and operable to lower density of the network structure when placed in contact with fluid and to thereby enable floating of the network structure.

In some embodiments, the capsule further comprises an actuator element placed within the cavity configured and operable to actively detach the at least one expandable element from the cavity on demand by a user, to thereby enable active evacuation of the capsule at any desired time.

In some embodiments, at least two different portions of the outer surface are at least partially made of two different materials surrounding different portions of the cavity, each material being configured to dissolve at a different region respectively, such that at least two expandable elements, each being located in a different portion of the cavity, successively expand at different regions of the gastrointestinal tract.

In some embodiments, the capsule may be configured for drug delivery and may be a passive slow release system. Alternatively, the capsule comprises a pump for delivering the drug substance. The pump may be based on different technologies including infusion pumps, peristaltic pumps, osmotic pumps, positive displacement pumps, and controlled release micropumps, like the MEMS piezoelectric micro pump or others.

In some embodiments, the capsule comprises a storage element configured and operable to contain the drug substance, such that the location of delivery of the drug can be controlled. The capsule may thus may configured as a controlled drug releasing device storing and delivering precise doses of therapeutic drugs or medicines into the stomach site, where it diffuses into the bloodstream.

In some embodiments, the capsule further comprises an RFID element placed within the cavity and is configured and operable to monitor a location of the capsule.

According to another broad aspect of the present invention there is provided a capsule comprising inter alia an operation generator configured and operable to controllably activate the capsule at different operation modes (e.g. a change in the electrostimulation pattern) with respect to the different zones of the gastrointestinal tract in which the capsule is located (which it passes through). To save energy, the capsule may also not be activated in some zones of the gastrointestinal tract. The different operation modes correspond to different electrostimulation patterns having different signal forms. More specifically, the capsule comprises a cavity having an outer surface; at least two electrodes for applying at least one electrical signal at at least one region of the gastrointestinal tract through which the capsule passes, the electrodes being located at the outer surface; an identification module placed within the cavity and configured and operable to identify different regions along the gastrointestinal tract by at least one of pH change identification and identification of electric impedance gradient; and an operation generator placed within the cavity and being connected to the at least two electrodes and to the identification module; the operation generator being configured and operable to carry out at least one of generating a signal at a specific operation mode having a certain electrostimulation pattern, modifying the electrostimulation pattern for each operation mode respectively with respect to the different regions identified by the identification module, and switching off the signal. It should be understood that treatment changes with respect to location within the gastrointestinal tract. The different zones include: the stomach, the small intestine and the colon. In the stomach, electrostimulation is aimed at reducing appetite, and as a result losing weight. To this end, location of the capsule is identified by pH and/or impedance and/or pressure and/or temperature change/measurement. The identification of the capsule's location may be based on at least one of the mentioned parameters. The pH and impedance may be measured by (1) pH sensor(s) located on the capsule, (2) providing sensitive (e.g. enteric) coating on one of the electrodes of the capsule, and measuring the impedance signal, (3) identifying a change in the impedance between different zones (4) providing a gradient impedance. Operation of different modes of electrostimulation may be controlled by biofeedback. The capsule may also be used as a targeted drug delivery device for delivering personal dosage to different zones, and may also be used for constipation treatment.

In some embodiments, the at least one electrical signal generated by the operation generator and applied by at least two electrodes is configured for at least one of measuring properties of the at least one region stimulating the at least one region and delivers a drug substance to the at least one region.

In some embodiments, the identification module comprises a pH identification unit configured and operable to identify change in pH in the different regions of the gastrointestinal tract. The pH identification unit comprises an additional electrode connected to the operation generator and is at least partially coated by a material being sensitive to pH, and a measuring element configured to measure current passing through the additional electrode.

In some embodiments, the operation generator comprises an electrical energy storage unit and a pulse generator having at least two output connections connected to the at least two electrodes.

In some embodiments, the identification module comprises an impedance gradient measuring unit connected to the operation generator and configured and operable to measure an impedance gradient of media surrounding the at least two electrodes.

In some embodiments, the operation generator is configured and operable to carry out at least one of generating a certain electrostimulation signal having a certain pattern, modifying the electrostimulation pattern for each operation mode respectively, and switching off the electrostimulation signal.

In some embodiments, the electrostimulation pattern is configured to create at least one type of neuromodulation stimulation, stimulation activity of muscles and pacing stimulation, to thereby control time of evacuation of the capsule from the gastrointestinal tract.

In some embodiments, the different regions of the gastrointestinal tract in which the capsule is activated comprise small intestine, large intestine and stomach.

In some embodiments, the operation generator produces a stimulation modulation pattern configured to block muscle contraction to thereby control the time the capsule is maintained in each region of the gastrointestinal tract.

In some embodiments, the operation generator is controlled by biofeedback.

In some embodiments, the different operation modes are determined according to at least one of type and amount of food ingested by the subject which may optionally be determined by a sensor or by input provided by the subject.

In some embodiments, at least a portion of the outer surface is at least partially made with at least one material configured to dissolve at a certain region of the gastrointestinal tract, in which the capsule passes through, the material self-dissolves, and at least a portion of the outer surface thereby dissolves.

In some embodiments in which the capsule comprises a storage element configured and operable to contain the drug substance, the operation generator is configured and operable for controlling delivery parameters of the substance at any location along the gastrointestinal tract. The delivery parameters comprise timing of delivery, dosage of delivery and location of delivery.

According to another broad aspect of the present invention there is provided a system, comprising the capsule as described above, and a non-transitory computer-readable medium containing a distributed application with software runnable on at least one personal device of a user. The software comprises an executable code that runs on the personal device. The personal device may be one of a mobile phone, a tablet, and a personal computer. The personal device may comprise receiver circuitry configured and operable to receive and transmit external data or commands regarding operation of the capsule. Additionally, the receiver circuitry may be configured and operable to receive external data or commands regarding exercise time and/or energy use and/or calorie burn and/or milestones, from at least one of the Internet, social site, media site and computing system.

In some embodiments, the system further comprises a connecting device (e.g. repeater) wearable by the user coupled to the capsule and to the personal device configured and operable to connect between the capsule and the personal device. The connecting device may be one of smart glasses, a wearable camera, a smart watch, a wristband, chest band, belt, smart ear wear, gloves, a necktie, a smart carrying bag, a smart backpack, smart clothing and smart shoes.

In some embodiments, the distributed (e.g. mobile) application for obesity treatment is configured and operable to display at least one of instructional data and dietary advice.

In a specific and non-limiting example, the system of the present invention may include a mobile application (APP), which runs on a smartphone. It connects the user to social networks and helps him to lose weight, while dealing with this application on a smartphone and occupying himself mentally, tricking his brain. The system may include a connecting device acting as a retranslator in the form of a wristband, which connects the capsule (inside the body) and his smartphone (outside the body).

In some embodiments, the system further comprises a dietary supplement (e.g. food or liquid) to be ingested by the subject for increasing retention time of the capsule in a certain region of the gastrointestinal tract, such that capsule retention may be obtained for a longer duration in the region.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1D represent a novel capsule for obesity treatment which controllably remains within a patient's body for a certain period of time according to some embodiments of the present invention;

FIGS. 2A-2C represent another possible configuration of the novel capsule according to some embodiments of the present invention;

FIGS. 3A-3C represent another possible configuration of the novel capsule according to some embodiments of the present invention;

FIGS. 4A-4D represent another possible configuration of the novel capsule according to some embodiments of the present invention;

FIGS. 5A, 5B, 5D, 5E represent another possible configuration of the novel capsule according to some embodiments of the present invention; FIG. 5C is an enlarged schematic illustration of a stent-like expandable element;

FIGS. 6A-6D represent another possible configuration of the novel capsule comprising a folded network structure according to some embodiments of the present invention;

FIG. 7A represents the different regions of the gastrointestinal tract through which the capsule passes, and is activated according to some embodiments of the present invention;

FIG. 7B represents a functional description of a possible configuration of the electronic block diagram of the capsule in which the novel capsule of the present invention comprises inter alia an operation generator configured and operable to activate the capsule in different operation modes with respect to the different zones of the gastrointestinal tract in which the capsule is located according to some embodiments of the present invention;

FIG. 7C represents a functional description of a possible configuration of the electronic block diagram of the capsule;

FIG. 8 represents functional descriptions of a possible configuration of the electronic block diagram of the capsule in which the identification module comprises a pH identification unit according to some embodiments of the present invention;

FIG. 9A represents a possible configuration of the capsule having at least one portion of the outer surface being at least partially made with at least one material being configured to dissolve at a certain region of the gastrointestinal tract through which the capsule passes, according to some embodiments of the present invention;

FIG. 9B represents a functional description of a possible configuration of the electronic block diagram of the capsule in which the novel capsule of the present invention is configured as a controlled drug releasing device, according to some embodiments of the present invention; and

FIG. 10 represents a system according to some embodiments of the present invention.

### DETAILED DESCRIPTION OF EMBODIMENTS

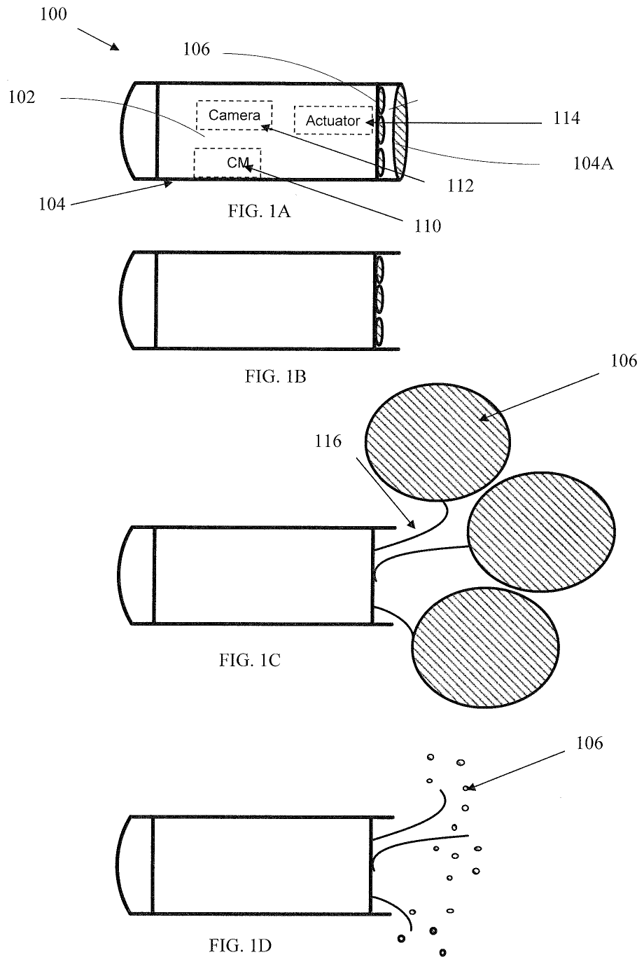

Reference is made to FIGS. 1A-1D representing a novel capsule 100 that controllably remains within a patient's body for a certain period of time. The capsule 100 comprises a cavity 102 having an outer surface (e.g. sealed housing) 104, and at least one expandable element 106 located within the cavity 102 in a non-expanded state, as illustrated in FIGS. 1A-1B.

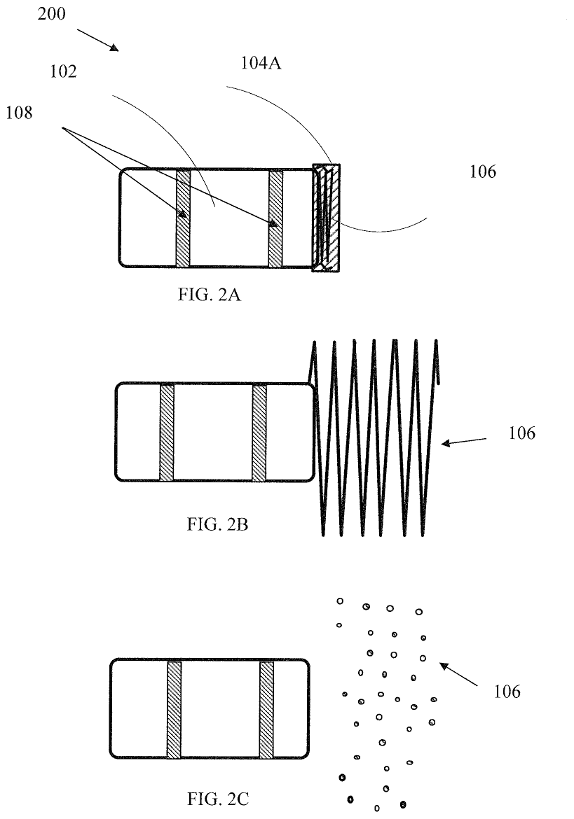

For the sake of illustration, three expandable elements 106 are represented, however the capsule 100 comprises at least one expandable element 106. Moreover, although for the sake of illustration the expandable elements 106 have a circular geometry, the expandable elements 106 are not limited to such configuration and can have the following non-limiting forms, including powders, particles, pellets, granules, spheres, beads, pills, balls, noodles, cylinders, extrudates and trilobes. In this example, the capsule 100 is a fully assembled capsule having cavity 102 with a compartment coated or made of a dissolving material (e.g. non-enteric, for example from hydroxypropyl methylcellulose (HPMC) or Gelatin) coating for gastric juice). Although in this specific and non-limiting example, the compartment is located at one side end of the capsule 100, its specific location in the cavity is not limited to any place and it can be for example also placed upside. It should be noted that location of the expandable element on the different parts of the cavity determines the orientation of the capsule. A plurality of expandable elements may be located at different spaced-apart positions on the circumference of the cavity. In this specific and non-limiting example, a portion of the outer surface 104A is made from a material being configured to dissolve at a certain region of the gastrointestinal tract, through which the capsule passes, such that the material self-dissolves and at least a portion of the outer surface is thereby dissolved. This stage is illustrated in FIG. 1B and enables exposition of the expandable elements with gastric juice. However, exposition to gastric juice may be obtained by using other mechanisms, as will be described further below. FIG. 1C shows a stage at which, after exposition to gastric juice, the expandable elements 106 configured to expand at a desired three-dimensional dimension upon exposition with gastric juice, are in an expanded state and are attached to a portion of the cavity 102 to thereby enable retention of the capsule 100 at the certain region. For example, if a polymer swelling ratio is about 30-50 in volume, a volume of about 30-50 ml can be represented in the form of a sphere having a diameter of about 3.8 cm to 4.6 cm which is large enough to provide gastric retention in a human stomach. The expandable elements 106 may be floating or non-floating units, and are aimed at holding the capsule 100 in the stomach. During the stage illustrated in FIG. 1C, after swelling of the expandable elements 106, the capsule is controllably maintained at a certain (e.g. fixed) location for a certain period of time. In this way, the capsule can controllably float in the stomach. In this specific and non-limiting example, the expandable elements 106 are attached to the cavity 102 by means of a bonding element 116. The bonding element 116 may be a wire or a suture or a glue. This configuration enables to independently control the disassembly and disintegration of each expandable element 106 by appropriately selecting the material of the bonding element 116 and/or by actively detaching each bonding element 116 from cavity 102 to independently control the disassembly of each expandable element 106. However, the expandable elements 106 may be directly attached to the cavity 102 as will be illustrated below in FIGS. 2A-2C. The expandable elements 106 are made of at least one material being configured for being evacuated after a certain period of time to thereby enable evacuation of the capsule outside the gastrointestinal tract as illustrated in FIG. 1D. In the non-limiting example described in FIG. 1D the expandable elements 106 are made of a material configured to self-disintegrate after a certain period of time. For example, the residuals of the expandable elements 106 may be in the form of a gel. The timing of each stage described in FIGS. 1A-1D is controllable by appropriately selecting the different materials (or a combination of different materials) of the different elements of the capsule. Although in the figure, the bonding elements 116 do not disintegrate, they can be made of a material which disintegrates after a certain period of time. The capsule 100 may also comprise an optional actuator element 114 placed within the cavity 102 configured and operable to actively detach the expandable elements 106 from the cavity 102 upon instructions/on demand by a user, to thereby enable active evacuation of the capsule 100 at any desired time. It allows active disassembly of the capsule 100 inside the gastrointestinal tract by a command of the user. For example, the actuator element 114 may be an electromechanical element such as a linear solenoid actuator.

The capsule 100 may comprise a communication module CM 110 powered by the same power supply such as a Radio Frequency Identification (RFID) element placed within the cavity 102 being configured and operable to receive and send any data relating to the operation of the capsule 100, such as data relating to the location of the capsule, commands, data relating to the activation and/or disintegration of the capsule, or data relating to the user nutrition parameters. The RFID may be a microchip placed inside the capsule cavity 102. It is thus possible to know whether the capsule 100 is still in the patient's body, or has left it. In this way, another capsule cannot be used if the first one is still inside a patient's body. The capsule 100 may also comprise a camera 112 configured for controllably imaging any desired region of the gastrointestinal tract, such that the location of the capsule can be controlled. In this embodiment, at least a portion of the capsule is made of a transparent material in order to define a certain field of view for the camera.

Reference is made to FIGS. 2A-2C representing another possible example of the configuration of the capsule 200 of the present invention. The elements are the same as described with reference to FIGS. 1A-1D except that the capsule 200 may comprise at least two electrodes 108 for applying at least one electrical signal at at least one region of the gastrointestinal tract through which the capsule 100 passes. However, the capsule of the present invention is not limited to the use of electronics. The electrodes 108 may be connected to a power supply (not shown) located in the cavity. The electrodes 108 may located at the outer surface 104 of the capsule. In this specific and non-limiting example, the at least one expandable element 106 is directly attached to the outer surface 104 of the capsule and when a portion of the outer surface 104A disintegrates, the expandable element 106 expands itself, and then, after a predetermined period of time, disintegrates into small particles (as shown in FIG. 2C). In this non-limiting example, the expandable element 106 is configured as a self-expandable biodegradable stent-like element, however the stent-like element may also be non-degradable (e.g. bare stents). The outer portion of the capsule 104A holds the stent-like expandable element 106 within the cavity 102 until its dissolution. When the outer portion of the capsule 104A is dissolved, as illustrated in FIG. 2B, the stent-like expandable element 106 opens itself.

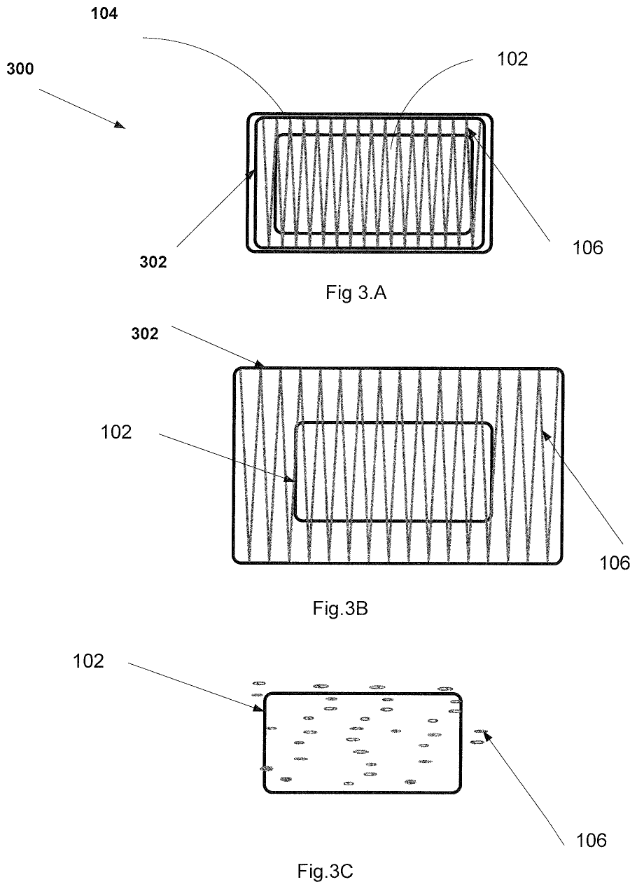

Reference is made to FIGS. 3A-3C representing another possible example of the configuration of the capsule 300 of the present invention. The elements are the same as described with reference to FIGS. 1A-1D except that the outer surface 104 of the capsule is spaced apart from the cavity 102 and is entirely made of a dissolving material configured to disintegrate after a certain period of time. In this example, the expandable element 106 is also configured as a stent-like element placed around the cavity 102. In this non-limiting example, the stent-like expandable element 106 is coated by protective layer 302 in order to prevent wounds by a bare stent while it remains in the stomach. This protective layer 302 may be at least partially made of material containing a flexible/stretchable biodegradable component. After disintegration of the dissolving material of the outer surface 104, as illustrated in FIG. 3B, the expandable element 106 expands itself, stretches protective layer 302 and then after a predetermined period of time both protective layer 302 and the expandable element 106 disintegrate into small particles as illustrated in FIG. 3C.

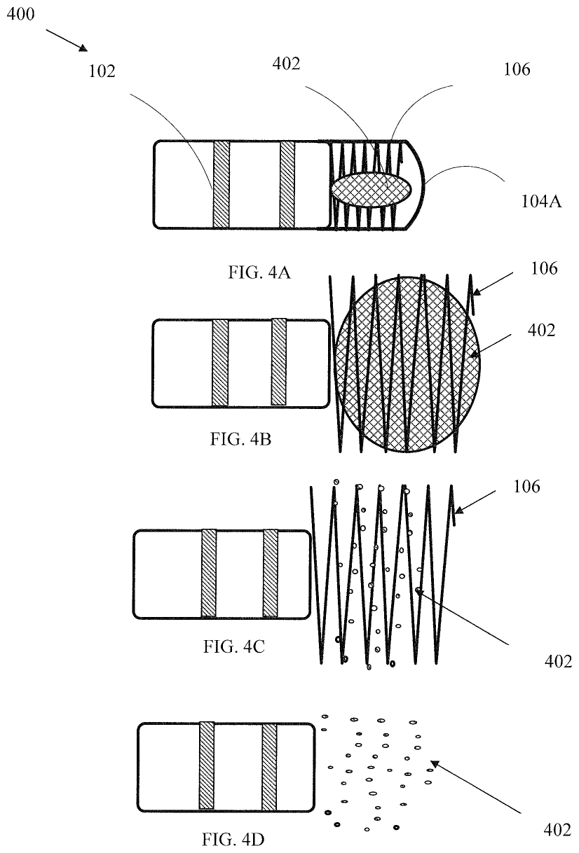

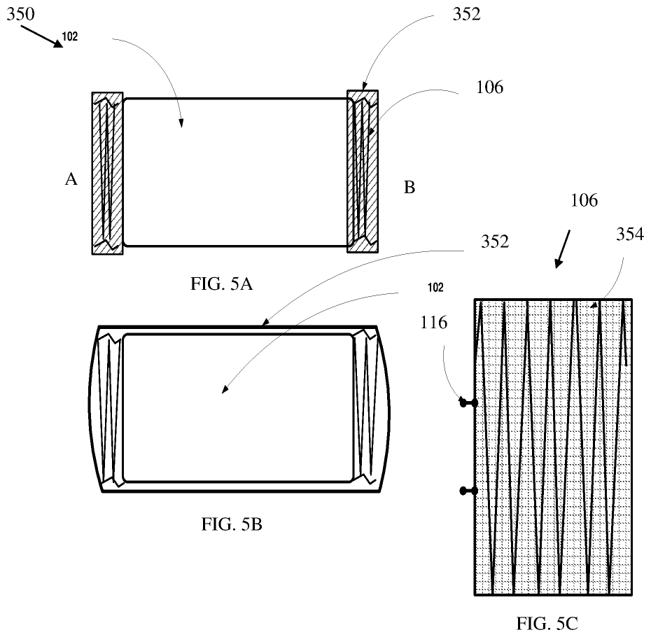

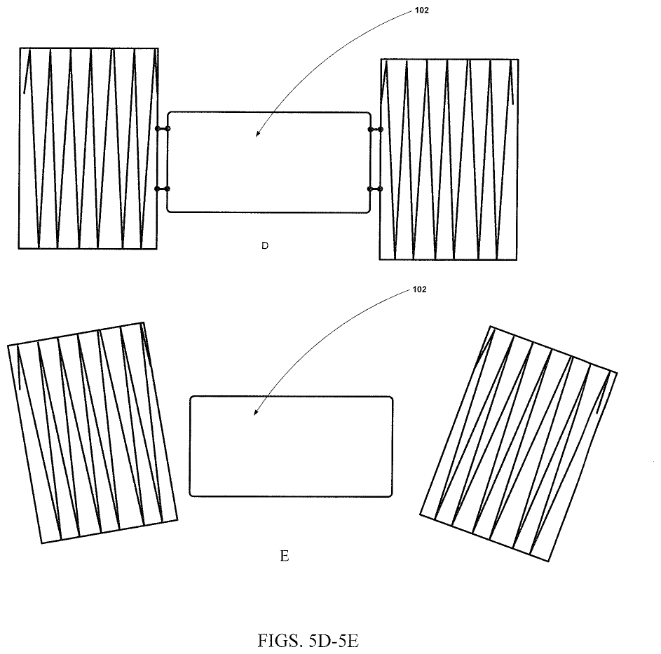

Reference is made to FIGS. 4A-4D representing another possible example of the configuration of capsule 400 of the present invention. The elements are the same as described with reference to FIGS. 1A-1D except that the expandable element 106 has a portion 402 made of a polymer-based material being capable of self-disintegration after a certain period of time. In this example, the expandable element 106 is also configured as a stent-like element placed at one edge of the cavity 102. For example the polymer-based material may be a superabsorbent polymer. Therefore in this example, the polymer portion 402 of the expandable element 106 unfolds it while it swells. After the portion of the outer surface 104A is dissolved as illustrated in FIG. 4B, the polymer portion 402 starts to press-crimp the stent-like expandable element 106 until it reaches its predetermined/desired three-dimensional dimension. Then, as illustrated in FIG. 4C, the polymer portion 402 dissolves, and the stent-like expandable element 106 holds the capsule 400 in the region of the gastrointestinal tract. In FIG. 4D, the stent-like expandable element 106 dissolves as well. However, the stent-like expandable element 106 may also not dissolve and may be evacuated outside the gastrointestinal tract. In this case the expandable element 106 is coated by a protective layer as described below with respect to FIGS. 3A-3C in order to prevent wounds by a bare stent while it remains in stomach.

Reference is made to FIGS. 5A-5E representing another embodiment of the present invention in which capsule 350 comprises a plurality of stent-like expandable elements 106. In this specific and non-limiting example of FIG. 5A, the capsule 350 comprises a cavity 102 having an outer surface having two different portions A and B located at the edges of the capsule 350 made by a dissolving material 352 being configured to disintegrate at a certain region of the gastrointestinal tract upon exposition with gastric juice, thereby disintegrating into portions A and B of the outer surface. In another specific and non-limiting example of FIG. 5B, the capsule 350 comprises a cavity 102 having an outer surface entirely made from a dissolving material being configured to disintegrate at a certain region of the gastrointestinal tract upon exposition with gastric juice, thereby disintegrating the entire outer surface. The dissolving material may comprise gelatin or an HPMC based material (e.g. capsules of the company CAPSUGEL may be used for this purpose). The largest capsule size is selected in accordance with a conventional empty capsules size chart. FIG. 5C is an enlarged schematic illustration of a stent-like expandable element 106 being in this example non-degradable (e.g. bare stents) and being enclosed in or coated by a protective layer 354 (e.g. plastic-made sheets/tubes). The stent-like expandable element 106 may be expanded by means of its radial forces inside stomach fluid, or may have openings through which gastric fluid can penetrate. The protective layer 354 is configured for preventing wounds by the stent-like element 106. The stent-like expandable element 106 may be connected to the cavity 102 by means of dissolvable bonding elements 116, for example biodegradable sutures at any portion of the cavity 102. When biodegradable sutures 116 dissolve, expandable elements 106 are released and separated from the cavity 102 as illustrated in FIG. 5D (assembled after expanding) and in FIG. 5E (after disintegration). It should be noted that each part of the disintegrated capsule, has a size equal to that which is presently standard, based on the size of endoscopic capsules, such as Given Imaging capsules in the range of about 26-32 mm length and 9-11 mm diameter.

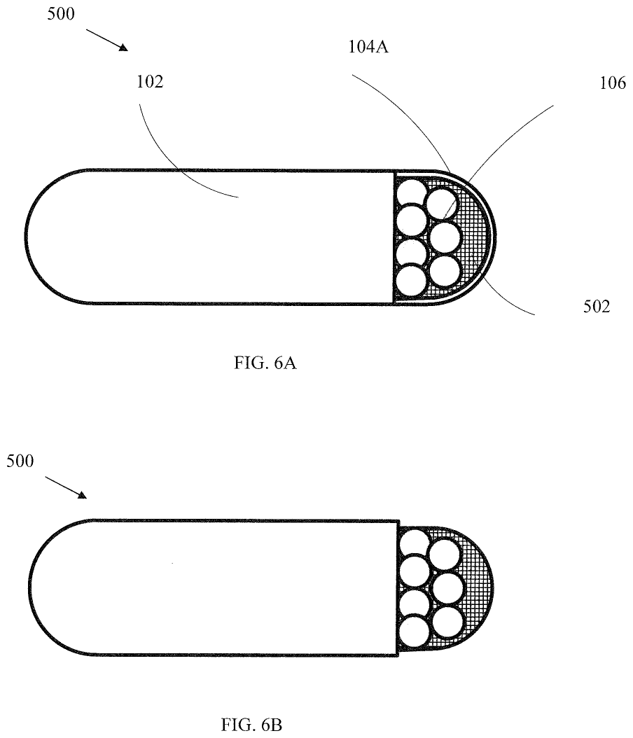

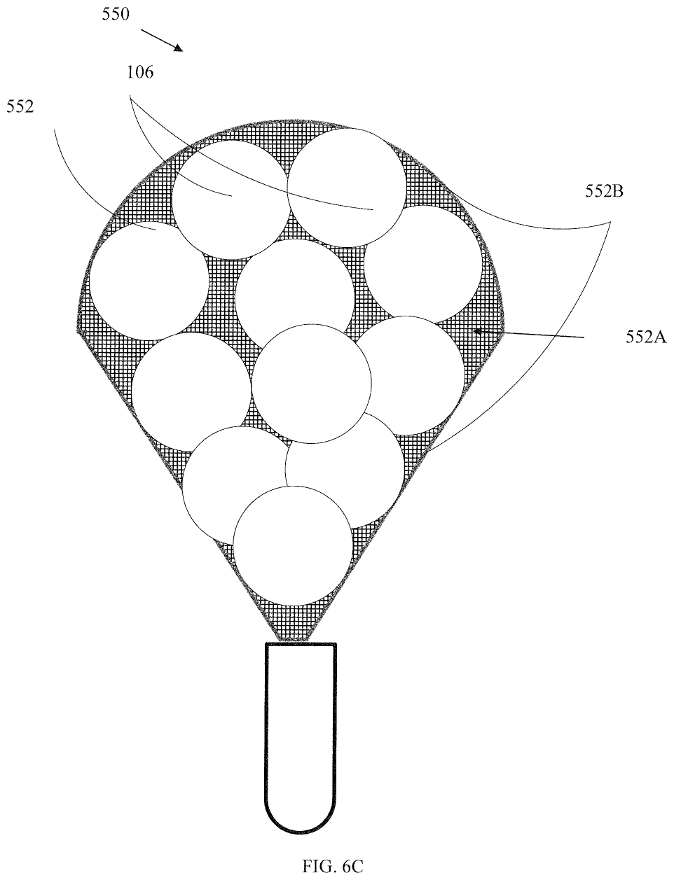

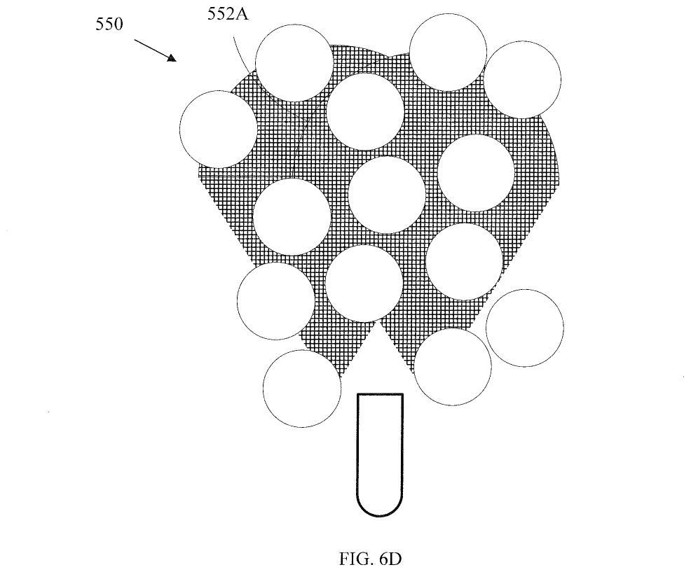

Reference is made to FIGS. 6A-6D representing another embodiment of the present invention in which the capsule 500 further comprises a folded network structure (e.g. mesh, net, bag, sheet, pouch, scaffold) 502 having openings. The dimensions of the expandable elements define the dimension of openings. A plurality of expandable/swelling elements 106 may be made of swelling polymers and may form a swelling unit. The shape of the swelling unit and of the expandable element depends on the shape of the network structure 502 and its mechanical strength is an integral function of mechanical strengths of the expandable elements 106 and the network structure 502. In this connection, it should be understood that to be able to operate as a gastric retention system, the capsule 500 should have a certain mechanical strength defined as strong enough for overcoming the gastric forces and not being evacuated by the stomach. The mechanical strength of the capsule overcomes forces of about 10 N. If the capsule were soft, the natural gastric forces would evacuate the capsule. However, a certain mechanical strength cannot be obtained with polymer material. The folded network structure enables, on one end, providing a certain mechanical strength to the capsule to permit its gastro retention, and, one the other end, enables penetration of the gastric juice within the structure through the openings to expand the expandable element while keeping the expandable element within an enclosed structure. The network structure 502 is configured for holding the expandable elements 106 placed therein. A dimension of the elements 106 is higher than a dimension of the openings such that, in a folded state, the elements 106 are not able to escape from the network structure 502. Although not illustrated, the openings/pores of the network structure 502 may have dimensions of different sizes. FIG. 6A shows the expandable elements 106 packed into the network structure 502, which is folded inside a compartment of the cavity 102 before swallowing. FIG. 6B shows that after the capsule 500 enters the stomach, upon disintegration of a portion of the outer surface 104A and exposition with gastric juice, the expandable elements 106 being in the specific example in the form of beads, start swelling, expand at a desired three-dimensional dimension, and unfold the network structure 502. FIG. 6C shows how the expandable bead elements 106 have swelled and fully unfold and stretch the network structure 502. Although not shown in the figures, the network structure may be made of a material configured for self-disintegration, such that the expandable elements are released from the network structure and are free to leave the stomach. The actuator element 114 of FIG. 1A may also mechanically push/pull a part of the expandable elements 106 connected to the cavity 102 and dissemble it from capsule 100 in a way which opens the network structure 502.

In some embodiments, the network structure and/or the expandable elements may be made of polymer-based material. The polymer-based material may be capable of being transformed to at least one of gel, hydrogel and suspension upon exposition with gastric juice. The polymeric formulation of the expandable elements may transform to gel from a dry state, when in contact with gastric juice, and may fully fill the network structure.

In the specific and non-limiting example, in which the capsule may comprise the at least two electrodes (not shown), the polymer-based material may be conductive polymer composites in order to provide good electrical contact with the internal part of the stomach and/or with the other parts of the GI tract and to enlarge the surface of the electrodes.

Table 1 below provides possible non-limiting examples of formulation components that may be used as a polymer-based material.

TABLE 1
Formulation Components (Weight, %)

| | Formulation no. | | | | | | |
|---|---|---|---|---|---|---|---|
| Excipient | 1 | 2 | 3 | 4 | 5 | 6 | 7 |
| Carbopol 974 | 75 | 10 | 50 | 35 | 30 | 20 | 10 |
| Sodium starch | 10 | | | 20 | | 10 | 20 |
| glycolate | | | | | | | |
| HPMC (K100M) | 10 | 10 | | 10 | | 25 | |
| Tannic acid | 5 | 5 | | | | 5 | |
| Crosscarmellose | | 75 | 10 | | 30 | 20 | 20 |
| sodium | | | | | | | |
| Conjuk glucomanane | | | 40 | 30 | 30 | | 30 |
| Cellulose acetate | | | | | 10 | | 10 |
| phthalate | | | | | | | |
| Guar gum | | | | | | 5 | 10 |
| Xanthan gum | | | | | | 10 | |
| Ethyl cellulose | | | | 5 | | 5 | |
| Total | 100 | 100 | 100 | 100 | 100 | 100 | 100 |

The polymer-based material may have at least one of the following properties: conductive and magnetic. For example, the polymer-based material may comprise magnetic particles configured for providing magnetic fixation of the capsule from the outside.

Although not shown, the capsule 500 may comprise one or more gas generating elements having effervescent properties (e.g. sodium bicarbonate) placed within the network structure 502 together with the expandable elements 106 and configured and operable to produce gas when placed in contact with fluid, to thereby increase inflation of the expandable elements 106. The capsule 500 may also comprise a filler (not shown) placed within the network structure 502 together with the expandable elements 106 configured and operable to lower density of the network structure 502 when placed in contact with fluid and to thereby enable floating of the network structure 502. The filler may be hollow or glass microspheres put inside the network structure 502 together with the expandable elements 106 made of polymer-based material in order to lower overall density of the network structure 502 with the expandable elements 106, which make it float.

Reference is made to FIG. 6C illustrating one embodiment of the present invention in which the network structure 552 may comprise at least two different materials configured to evacuate the gastrointestinal tract after a certain period of time. At least one of the materials may be configured to dissolve at a certain region of the gastrointestinal tract to thereby enable escape of the expandable elements 106. Such materials may be made from at least one of polymer and biological sutures such as plain and chromic catgut. In the example of FIG. 6C, capsule 550 comprises a network structure 552 made of two types of textile/suture defining one non-dissolvable portion 552A and one dissolvable portion 552B. Therefore, the capsule 550 may disintegrate in three different elements: the cavity 102, the empty network structure 552 and the expandable elements 106. In this example, the network structure 552 comprises the network itself 552A made of a non-dissolvable material surrounded by dissolvable sutures 552B. The network structure 552 may therefore be at least partially made of at least one of stretchable material, conductive material (e.g. textile metalizing silver coated fabrics) and biological sutures. In this specific and non-limiting example, the capsule 550 may comprise the at least two electrodes (shown for example in FIG. 1A) for applying at least one electrical signal at at least one region of the gastrointestinal tract in which the capsule passes through, the electrodes being located at the outer surface. However, the invention is not limited to the use of electrodes. If the network structure 552 is at least partially made of a conductive material, good electrical contact with the internal part of the stomach and/or with the other parts of GI tract is provided and the surface of the electrodes can be enlarged.

If the network itself 552A is made of a polymer-based material, the transformation of the polymer into gel enables retention of the expandable elements 106 within the network structure 552. In this specific example, after a specific period of time, as illustrated in FIG. 6D, the sutures 552B of FIG. 6B are dissolved, the network 552A unfolds and the expandable elements 106 are freed to leave the stomach naturally. The rest of network 552A leaves the stomach as well. Alternatively, if the expandable elements 106 are not transformed into gel, after a certain time period the expandable elements 106 are broken into small particles and thus self-disintegrate, and the particles are able to escape via the openings of the network structure 552 and the network itself 552A also disintegrates after a certain period of time.

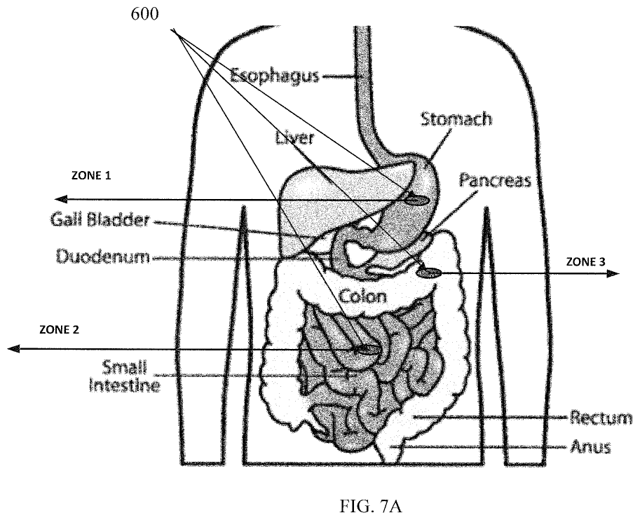

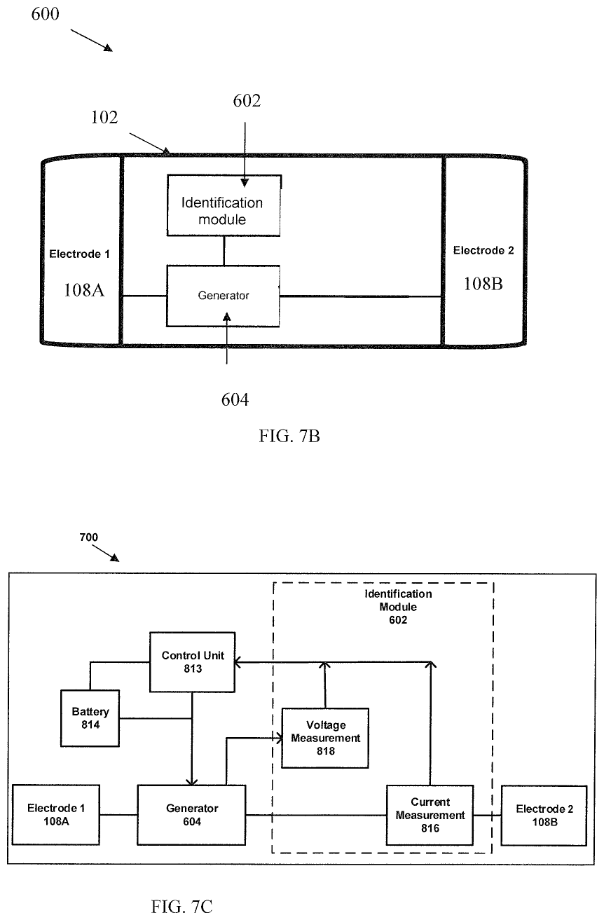

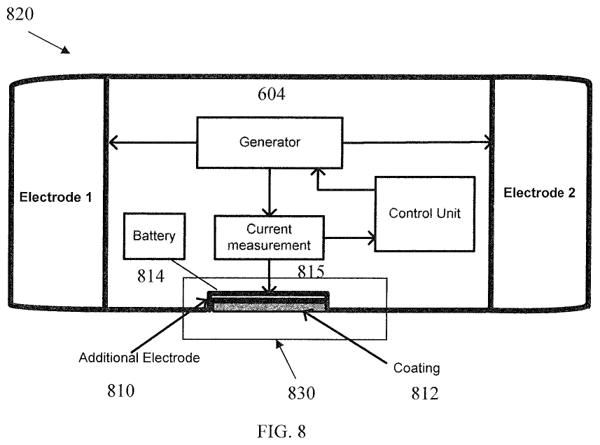

Reference is made to FIG. 7A, illustrating the different regions of the gastrointestinal tract through which the capsule passes and is activated, denoted respectively as Zone 1, Zone 2 and Zone 3. The number of the zones through which the capsule 600 passes is not limited. In this specific example, the different zones of stimulation include: the stomach (Zone 1), the small intestine (Zone 2) and the colon/large intestine (Zone 3). In each zone, different or the same type of stimulation can be used based on the known location of the capsule 600. A different number of types of Gastric Electrical stimulations (GES) can be used in the different zones, such as long pulses, short pulses and trains of short pulses. Pulses may be defined as a series of bipolar pulses with a constant current or constant voltage. The parameters of these pulses which could be varied include frequency, pulse width and amplitude (usually up to 10 mA). It is clinically approved, that using GES in the stomach (Zone 1) with trains of short pulses, increases the feeling of satiety with subsequent reduced food intake, weight loss and reduction of appetite. Gut electrical stimulation or Intestinal Electrical Stimulation (IES) could be applied in the different zones, and in particular in Zone 2 and Zone 3. The types of electrical stimuli of IES are similar to GES, yet currently there are no implantable devices available on the market for IES. Dual pulses (combinations of short and long pulses) and Synchronized Stimulation (using two pairs of electrodes: one for detection of intestinal slow waves and the other for stimulation) may be used to operate IES, and may reduce food intake via inhibition of gastric emptying, acceleration of intestinal transit and/or reduction of fat absorption. IES also may decrease hunger hormone, ghrelin, and increase satiety hormone, cholecystokinin. It also acts on vagal neuronal mechanisms. The location detection is based on measurements of physiological parameters (e.g. pH, impedance, impedance gradient, temperature, pressure). The location of the capsule 600 may thus be identified by pH and/or impedance and/or pressure and/or temperature change/measurement. The pH and impedance may be measured by (1) pH sensor(s) located on the capsule, (2) providing sensitive (e.g. enteric) coating on one of the electrodes of the capsule, and measuring the impedance signal, (3) measuring of change in the impedance between different zones (4) providing a gradient impedance as will be illustrated further below with respect to FIGS. 7C and 8. Although not shown, the capsule 600 may be at least partially made from at least two different materials surrounding different portions of the cavity. Each material is configured to dissolve at a different region respectively, such that at least two expandable elements, each being located in a different portion of the cavity, successively expand at the different regions of the gastrointestinal tract.

Reference is made to FIG. 7B, illustrating an embodiment of the present invention in which the capsule 600 comprises a cavity 102, at least two electrodes 108A and 108B for applying at least one electrical signal at at least one region of the gastrointestinal tract through which the capsule 600 passes, and an identification module 602. Identification module 602 is placed within the cavity 102 and is configured and operable to identify different regions along the gastrointestinal tract by at least one of pH change identification and identification of electric impedance gradient, and operation generator 604 also placed within the cavity 102 and being connected to the at least two electrodes 108A and 108B and to the identification module 602. Operation generator 604 is configured and operable to activate the capsule 600 in different operation modes (e.g. change the electrostimulation pattern) with respect to the different zones of the gastrointestinal tract in which the capsule is located (through which it passes). In particular, the operation generator 604 may be configured and operable to generate a signal having a certain electrostimulation pattern to be applied to the electrodes 108A and 108B and/or modify the electrostimulation pattern for each operation mode respectively and/or switch off the signal. It should be understood that treatment changes with respect to location within the gastrointestinal tract. The electrostimulation pattern may be preprogrammed as desired. The geometrical shape of the pulses is not limited to any shape and may be square, sinusoidal, triangular, or saw-tooth. The intensity of the signals of the electrostimulation pattern can be also varied according to the operation mode and/or of the location of the capsule. Operation generator 604 may be preprogrammed to activate the capsule at a certain time pattern. Additionally or alternatively, operation generator 604 may be connected to a remote control device (e.g. distributed (e.g. mobile) application) configured for selecting a different type of stimulation with respect to the time pattern and/or may receive via the communication module 110 of FIG. 1A or via a distributed (e.g. mobile) application the time pattern of activation. The electrostimulation pattern may be configured to create at least one type of neuromodulation stimulation, stimulation activity of muscles and pacing stimulation, to thereby control time of evacuation of the capsule from the gastrointestinal tract. The operation generator 604 may produce a stimulation modulation pattern configured to block muscle contraction to thereby control the time the capsule 600 is maintained in each region of the gastrointestinal tract. In the stomach and small intestine, the operation generator 604 may use special muscle blocking stimulation in order to prevent capsule expulsion. Two types of stimulation may be used simultaneously: one for push out prevention, and one for stimulation. The different operation modes correspond to different electrostimulation patterns having different signal forms. For example, the signal forms may comprise two stimulation types: one for simulation of nerves, and one for muscles, which delay capsule removal from stomach and intestine. The different kinds of stimulation used include also gastric delay, acceleration, vagal blocking etc. The electrical stimulation via electrodes 108A and 108B may therefore be configured and operable to stimulate a region in the gastrointestinal tract, block muscle contraction or neurostimulation for vagal block therapy, which may delay or stimulate activity in the intestine, or hold the capsule for a longer duration in the stomach and/or intestine. Different types of electrical stimuli may be used in Gastric Electrical Stimulations (GES) including long-pulse, short pulse and trains of a short pulses. The present invention is not limited to any type of electrical stimuli. The operation generator 604 may be programmed respectively. For example, one electrostimulation pattern may be a high energy stimulation pattern which consists of pulses at a low frequency, a few cycles per minute (CPM), just above the normal electrical frequency of the stomach and having a duration in milliseconds (e.g. 200 ms-300 ms). This pattern can pace the organ involved. Another electrostimulation pattern may be a gastrointestinal neural electrical stimulation pattern for affecting contractions which consist of high-frequency trains of pulses, bipolar waves with a duration of about 20 ms with high amplitude. Yet another type of pattern may be a low energy stimulation pattern producing trains of square wave pulses at a high frequency (Hz), having a duration in microseconds, and amplitude in the range of a few mA. Such a specific pattern produces neuromodulation. It should be noted that these electrostimulation patterns are generally known in the art, as described for example in the article entitled “Gastric Electrical Stimulation for Obesity” published in Current Gastroenterology Reports 17(1):424, January 2015.

Reference is made to FIG. 7C showing a functional description of a possible configuration of the electronic block diagram of the capsule 700 in which the identification module 602 is implemented as an impedance gradient measuring unit connected to the operation generator 604 and configured and operable to measure an impedance and an impedance gradient of media surrounding the at least two electrodes 108A and 108B. In the figure, the electronic block diagram of the capsule 700 comprises inter alia the two electrodes 108A and 108B, the operation generator 604 which may comprise an electrical energy storage unit 814 (referred as battery) and a pulse generator (not shown) generating pulses and having at least two output connections connected to the two electrodes 108A and 108B. In this specific and non-limiting example, the identification module 602 includes a voltage measurement unit 818 and a current measurement unit 816. The voltage measurement unit 818 and the current measurement unit 816 measure the voltage and the current of the media surrounding the at least two electrodes 108A and 108B, respectively. The control unit 813 receives the results of voltage measurements and current measurements and determines the impedance of the media surrounding the at least two electrodes and the impedance gradient of the media surrounding the at least two electrodes. The control unit 813 processes the impedance gradient to determine the location of the capsule. The location of the capsule is then provided to the operation generator 604 which activates specific stimulation pattern via the electrodes 108A and 108B accordingly. Identification module 602 includes a voltage measurement unit 818 and current measurement unit 816. More specifically, control unit 813 comprises a signal processor adapted to calculate the impedance gradient and correlate the impedance gradient with the location of the capsule. More specifically, the impedance gradient is calculated as the impedance difference at certain time points, for example 10 sec. For example, impedance gradient (GImp), may be calculated on a base of the below formulae:

$$GImp = \frac{{Imp}_{2} - {Imp}_{1}}{Δ t} ;$$

Here Imp₁ and Imp₂ are successive impedances calculated from data measured at the time points t₁ and t₂, thus Δt=t₂−t₁. The signal processor may comprise a data input utility including a communication module for receiving the impedance signal and transmitting a signal to the operation generator indicative of the time pattern and type of stimulation, an optional data output utility for generating data relating to the location of the capsule, a memory (i.e. non-volatile computer readable medium) for storing database i.e. preselected data indicative of impedance gradient versus the location of the capsule, and a data processing utility adapted for calculating the impedance gradient and selecting the corresponding type of stimulation and time pattern. The database may be implemented with Microsoft Access, Cybase, Oracle, or other suitable commercial database systems. The signal processor is configured in a cloud-based configuration and/or utilize Internet based computing so that parts of processing utility, and/or memory may reside in multiple distinct geographic locations. The processor may query/cross-reference the received data with data in the database to locate the capsule and may communicate such location data to a mobile device at which the processor may signal to display a message corresponding to the location data. To this end, the preselected data stored in a database may be used to compare the measured impedance gradient with the stored impedance gradient. The memory may be relayed via wireless or wired connection by an external unit to a central database.

Table 2 below provides examples of different impedances corresponding to different locations in the GI tract.

TABLE 2

| | Conductivity, | Impedance for sample |
|---|---|---|
| Part of GI Tract | 10E−3*(1/cm*ohm) | conductor, ohm |
| Gastric Juice | 23 | 217 |
| Stomach | 7.5 | 660 |
| Small Intestine | 10 | 500 |
| Colon | 6 | 830 |

For example, if control unit 813 obtains small values (+/−10-20% of sample conductor impedance level from Table 2) of GI, control unit 813 correlates the location of the capsule to be still in the same GI zone. If control unit 813 obtains peak data greater than 10 ohm/sec, control unit 813 correlates the location of the capsule to be in the next region (from stomach to small intestine or from the small intestine to colon). For example, the system may comprise a timer measuring the time elapsed from the insertion of the capsule into the body. It is generally known that if about 6-8 hours have passed, the capsule is probably still in the stomach. After about 8 hours, the capsule would probably be in the colon. By using the correlation data of Table 2 above, the measurement of the absolute value of the impedance is correlated with the location of the capsule. The measurement of the impedance gradient enables evaluating the passage of the capsule from one zone to another. If the impedance gradient has no significant change, the capsule is still in the same gastrointestinal zone. A peak in the impedance gradient indicates that the capsule has moved from one zone to another.

In some embodiments, identification module 602 identifies a capsule location by identifying a change in impedance. Such a change may be identified for example as a difference in impedance between the stomach, where the capsule is located in the proximity of mucosa, or floats in gastric juice, and the interior of narrow intestines, where it is in close contact with the inner intestine lumen. In this way, the identification module 602 correlates the change in impedance to the change in location through which the capsule passes. The identification algorithm may be based on a sum of several factors: real transient time (i.e. the period of time in which the capsule is retained in a body since the moment of capsule activation), impedance, gradient of impedance, and database correlating between the impedance and the location of the capsule (e.g. Table 2). By using the correlation data from Table 2 and the measured impedance, it is possible to estimate where the capsule may be located.

In some embodiments, the capsule is configured for using impedance measurements for non-direct measurements of pH. The database also comprises a look up table in which pH can be calculated based on impedance measurements according to the following formula: pH=K*Imp, wherein K is calculated from calibration measurements. The calibration measurements of impedances are obtained for liquids having different values of pH. Control unit 813 may thus also provide pH values to enable to correlate between the impedance measurements to the location of the capsule.

In some embodiments, the capsule may be configured and operable to be activated according to a certain meal plan/schedule provided by the user or by an advisor. The capsule is then activated at a certain time pattern in accordance with the time of meals. For example, the user sets a time of food intake. The capsule may receive via the communication module 110 of FIG. 1A or the distributed (e.g. mobile) application the meal plan of a specific user. Operation generator 604 may activate the capsule about thirty minutes before the first meal, to provide a feeling of satiety before the ingestion of the meal. The type of stimulation may be different before or after each meal. After a certain time period, the capsule may be deactivated (i.e. switched off) until the next meal, when the sequence is repeated again. Any one of the methods for controllably evacuating the capsule as described above after a certain period of time before the next meal (e.g. 3 hours), may be implemented.

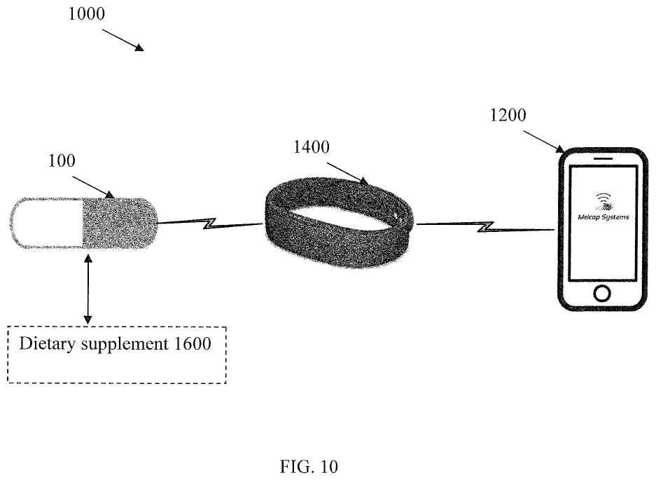

As described above, the user has the possibility to change parameters of the stimulation pattern and even to switch it off totally, when he/she feels any discomfort. The parameters can be changed using a mobile application or any other remote control unit. The parameters include at least one of current level (i.e. amplitude of signal of the stimulation pattern, frequency of the stimulation pattern and treatment duration). For the sake of safety, the current level has a maximal threshold. For example, it may be defined as not exceeding 8 mA. Several possible actions may be taken in order to stop treatment/stimulation. For example, a user may stop the treatment by using one of three options: via mobile application (e.g. by choosing “stop treatment” command), via a connecting device such as a repeater, as will be described below with respect to FIG. 10. The repeater may comprise a stop/continue button. Alternatively, the user may remove the repeater from any part of his/her body e.g. hand, head. In this case, connection is lost and the treatment is interrupted. Operation of the different modes of electrostimulation may be also controlled by biofeedback given by the user.

The database may also comprise data correlating at least one of type and amount of food ingested by the subject which may optionally be determined by a sensor (not shown), or by input provided by the subject with the different types of stimulation.

Reference is made to FIG. 8 showing a functional description of another possible configuration of the electronic block diagram of the capsule 820 having an identification module comprising a pH identification unit 830. The pH identification unit 830 is configured and operable to identify a change in pH in the different regions of the gastrointestinal tract. The pH identification unit 830 comprises an additional electrode 810 connected to the operation generator 604 and is at least partially coated by a material 812 being sensitive to pH and a measuring element 815 configured to measure current passing through the additional electrode 810. Therefore, the outer surface of the additional electrode 810 is covered by a special coating 812 configured to enable current detection and measurement only after its dissolving. The special coating 812 may be an enteric coating, which starts to dissolve only in the small intestine area having a pH higher than 4, while the capsule leaves the stomach filled by acid gastric fluid with low pH (1-3). The additional electrode 810 is connected to the positive contact of the electrical energy storage unit (e.g. battery) 814 and the current passing through the electrode 810 is measured by a current measurement unit 815, the output of which is connected to the control input of the operation generator 604.

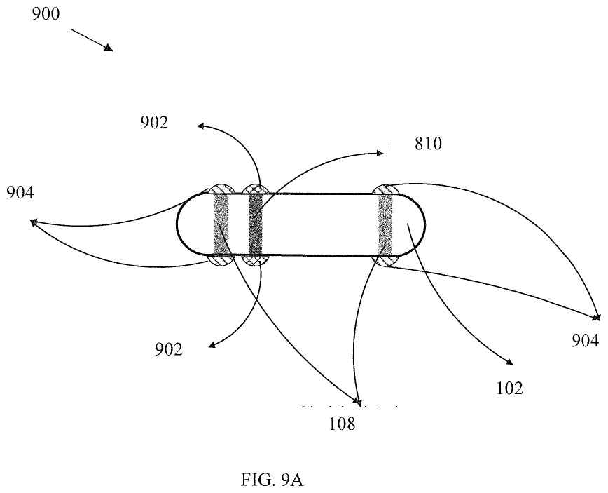

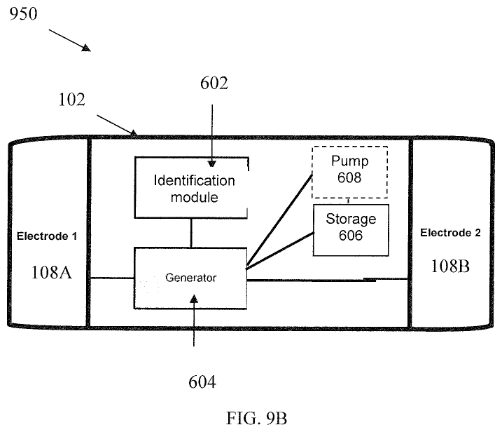

Reference is made to FIG. 9A showing an embodiment in which the capsule 900 has at least one portion of the outer surface 904 being at least partially made with at least one material being configured to dissolve at a certain region of the gastrointestinal tract through which the capsule 900 passes. In this specific and non-limiting example, the portions of the outer surface 904 are at least partially made from at least one material configured to dissolve at a certain region of the gastrointestinal tract, the portions covering the electrodes 108 being made from a non-enteric (e.g. gelatin) coating. The non-enteric coating dissolves in the gastric juice to expose the electrodes 108. Moreover, in this example, the capsule 900 also comprises an additional electrode 810 as described with respect to FIG. 8 above, being a part of the pH identification unit as described above, and being at least partially coated by a material 902 being sensitive to pH. Therefore, the portion of the outer surface 902 covering the additional electrode 810 is covered by an enteric coating configured to enable current detection and measurement only after its dissolving.

Reference is made to FIG. 9B showing an embodiment in which the capsule 950 may also be used as a targeted drug delivery device for delivering personal dosage to different zones. In this specific and non-limiting example, the capsule 950 comprises a sealed housing with two built-in electrodes 108A and 108B located at the outer surface of the cavity 102, operation generator 604, identification module 602 and a storage element 606 configured and operable to contain a drug substance. The targeted drug delivery device may be a passive slow release system. Alternatively, capsule 950 may also comprise a pump 608 connected to storage element 606 and configured for delivering the drug substance. The pump may be based on different technologies including infusion pumps, peristaltic pumps, osmotic pumps, positive displacement pumps, and controlled release micropumps, like MEMS piezoelectric micro pump or others.

The operation generator 604 is then also connected to the storage element 606 and optionally to pump 608 and is configured and operable for controlling delivery parameters of the substance at any location along the gastrointestinal tract. The delivery parameters comprise a timing of delivery, a dosage of delivery and a location of delivery. In this embodiment, the electrodes 108A and 108B are configured for inter alia delivering a drug substance to the at least one region. For example, the electrodes 108A and 108B may be configured to deliver a drug substance to the at least one region by using Electromotive drug administration (EMDA) or by Iontophoresis, such that location of delivery of the drug can be controlled. As used herein, the word “drug” refers to medicines, placebos, non-medicinal substances, contrast agents, gases, fluids, liquids, radiological agents, imaging or medical markers, sensors for monitoring the person's vitals, etc. Identification module 602 enables to determine the location of the capsule and to deliver the drug substance precisely in the region of interest (e.g. in the stomach).

Reference is made to FIG. 10 showing an embodiment in which there is provided a system 1000 comprising a capsule 100 as described above and a non-transitory computer-readable medium containing a distributed application with software runnable on at least one personal device of a user 1200. In this specific and non-limiting example, the system 1000 of the present invention may include a mobile application (APP), which runs on smartphone 1200. Although a smartphone is represented, the personal device of the user is not limited to such a device and can be any one of a mobile phone, tablet, and personal computer. The personal device 1200 comprises a receiver circuitry configured and operable to receive external data or commands regarding exercise time and/or energy use and/or calorie burn and/or milestones, from at least one of the Internet, social site, media site and computing system. It can therefore connect the user to social networks and assist him to lose weight while dealing with this application on a smartphone and occupying himself mentally, tricking his brain based on medical paradigm of Gut Brain Axis, known as GBA. The system 1000 also includes a connecting device 1400 acting as a retranslator or repeater in the form of a wristband, which connects the capsule 100 (inside the body) and his smartphone 1200 (outside the body). The connecting device may be wearable by the user coupled to the capsule 100 and to the personal device 1200, configured and operable to connect between the capsule 100 and the personal device 1200. For the sake of illustration only, a wristband is represented, however, the connecting device 1400 may be one of smart glasses, a wearable camera, a smart watch, a wristband, chest band, belt, smart ear wear, gloves, a necktie, a smart carrying bag, a smart backpack, smart clothing and smart shoes. The connecting device 1400 and the system 1000 comprise a respective network communication interface module for wireless communication, which can include a communication module (e.g. RF transceiver). The distributed (e.g. mobile) application for obesity treatment may be configured and operable to display at least one of instructional data and dietary advice. It should be understood that a device or a drug alone cannot help users to lose weight, therefore the present invention provides a distributed application that uses mental effects, such as occupying the user's brain, which helps the user to diet and control food intake. As described above, the type of stimulation used may depend on the type and/or amount of food intake. The type and/or amount of food intake may be one of the input of the distributed application to be filed by the user. The system 1000 may also further comprise a dietary supplement 1600 such as food or liquid (e.g. fiber or protein bars) to be ingested by the subject/user for increasing retention time of the capsule 100 and delaying migrating motor complex (MMC) in a certain region of the gastrointestinal tract such that longer capsule retention may be obtained in the region. In this way, the capsule 100 can be held for a longer duration in the stomach.

We claim:

1-18. (canceled)

19. A capsule comprising:
a cavity having an outer surface;
at least two electrodes for applying at least one electrical signal at at least one region of the gastrointestinal tract through which the capsule passes, the electrodes being located at said outer surface;
an identification module placed within said cavity and configured and operable to identify different regions along the gastrointestinal tract through which the capsule passes by at least one of pH change identification and measurement of electric impedance gradient; and
an operation generator placed within said cavity and being connected to said at least two electrodes and to said identification module; said operation generator being configured and operable to carry out at least one of generating a signal at a specific operation mode having a certain electrostimulation pattern, modifying the electrostimulation pattern for each operation mode respectively with respect to said different regions identified by said identification module, and switching off said signal.

20. The capsule of claim 19, wherein said generator generates at least one electrical signal via said at least two electrodes being configured for at least one of measuring properties of said at least one region stimulating said at least one region and delivering a drug substance to said at least one region.

21. The capsule of claim 19, wherein said identification module comprises a pH identification unit configured and operable to identify a change in pH in said different regions of the gastrointestinal tract, said pH identification unit comprising an additional electrode connected to said operation generator and being at least partially coated by a material being sensitive to pH and a measuring element configured to measure current passing through said additional electrode.

22. The capsule of claim 19, wherein said operation generator comprises an electrical energy storage unit and a pulse generator having at least two output connections connected to said at least two electrodes.

23. The capsule of claim 19, wherein said identification 5 module comprises an impedance gradient measuring unit connected to said operation generator and configured and operable to measure an impedance gradient of media surrounding said at least two electrodes.

24. The capsule of claim 19, wherein the electrostimulation pattern is configured to create at least one type of neurostimulation, stimulation activity of muscles and pacing stimulation to thereby control time of evacuation of said capsule from the gastrointestinal tract.

25. The capsule of claim 19, wherein said different regions of the gastrointestinal tract in which the capsule is activated comprise small intestine, large intestine and stomach.

26. The capsule of claim 19, wherein said operation generator produces a stimulation modulation pattern configured to block muscle contraction to thereby control the time the capsule is maintained in each region of the gastrointestinal tract.

27. The capsule of claim 19, wherein said operation generator is controlled by biofeedback.

28. The capsule of claim 19, wherein said different operation modes are determined according to at least one of type and amount of food ingested by said subject.

29. The capsule of claim 19, further comprising a sensor configured and operable to determine at least one of type and amount of food ingested by said subject.

30. The capsule of claim 19, wherein the determination of at least one of type and amount of food ingested by said subject is performed by at least one of said sensor and by input provided by said subject.

31. The capsule of claim 19, wherein at least a portion of the outer surface is at least partially made with at least one material being configured to dissolve at a certain region of the gastrointestinal tract, through which the capsule passes, said material self-dissolving, thereby dissolving at least a portion of the outer surface.

32. The capsule of claim 19, further comprising a storage element configured and operable to contain said drug substance, said operation generator being configured and operable for controlling delivery parameters of said substance at any location along said gastrointestinal tract; said delivery parameters comprise a timing of delivery, a dosage of delivery and a location of delivery.

33-38. (canceled)
